(19) United States
(12) Patent Application Publication
(10) Pub. No.: US 2009/0240210 A1
(43) Pub. Date: Sep. 24, 2009

(54) AUTO RETRACTABLE SYRINGE

(75) Inventors: Graeme Francis Walton, New South Wales (AU); Allan Walsh, New South Wales (AU)

Correspondence Address: BROOKS KUSHMAN P.C., 1000 TOWN CENTER, TWENTY-SECOND FLOOR, SOUTHFIELD, MI, 48075, US

(73) Assignee: GLOBAL MEDISAFE HOLDINGS LIMITED, The Hill, NSW (AU)

(21) Appl. No.: 11/908,682

(22) PCT Filed: Jan. 6, 2006
(86) PCT No.: PCT/US06/00331

§ 371 Date: Sep. 14, 2007

(30) Foreign Application Priority Data
Mar. 14, 2005 (AU) 2005901208
Aug. 2, 2005 (AU) PCT/AU2005/001132
Nov. 15, 2005 (AU) 2005906317

(51) Int. Cl. A61M 5/32

(52) U.S. Cl. 604/196

(57) ABSTRACT

An auto-retractable safety syringe comprising a syringe barrel (12) having a hollow plunger (13) slideably located therein. The needle assembly comprises a needle and a needle hub (17), with a groove in the end of the barrel (12) to hold the needle hub (17) and a spring disposed over said needle hub (17) and acting between the needle hub (17) and the end of the barrel (12). The needle hub (17) has a groove around its circumference in which a locking means mates so as to retain the needle hub (17) in the end of the syringe barrel (12) during use. The plunger (13) upon completion of an injection and when further depressed activates auto retraction of the needle hub (17) by dislodging said locking means from said groove to release the needle hub (17) hence allowing the needle and spring to retract inside the hollow plunger (13) under the influence of the spring.

### TECHNICAL FIELD

This invention relates to a safety syringe having an auto retractable needle for use in the medical or dental profession or in personal drug administration so that the physician, surgeon or other needle operator might be protected from injury by the needle after its use. The invention in one preferred form relates in particular to a gland assembly which facilitates auto retraction of a needle hub of the syringe upon completion of the injection. However the invention is not limited thereto, and any syringe in which the auto-retraction mechanism according to the present invention may also be applied is meant to be include.

### BACKGROUND

In recent times there has been growing evidence of needle stick injuries, whether from the medical profession or from children playing on the beach or in parks. No matter whether it involves the average man in the street, or professional health workers, the results are that someone will be infected with serious blood born diseases, as a result of receiving a needle stick injury.

The danger of injury and possible infection from the HIV or hepatitis B virus to medical practitioners using needles in the normal course of their business is well documented.

Furthermore, persons who are in the habit of administering drugs to themselves run a severe risk of contacting either of the specified viruses, or indeed contacting other viruses if a needle, once used, is reused in an un-sterilised form. There are also added dangers where the needle in the syringe is required to be replaced.

There have been many proposals aimed at reducing the number of so-called needle-stick injuries and various attempts have been made to provide a safe system for disposal of such needles once used, but such prior proposals have had deficiencies.

Although the following description refers primarily to the use of the invention in relation to a so-called exchange needle safety syringe where there is a requirement to accommodate various sizes of needles or where a needle change is required between filling the syringe from a vial and giving an injection to the patient, the invention is not limited thereto and the use of the invention in any syringe where it is desired to provide auto-retraction of the needle after completion of the injection is also contemplated. Thus any syringe requiring an auto retractable needle is meant to be encompassed, where, by suitable modification as necessary, the invention may also be utilised.

### OBJECT OF THE INVENTION

It is an object of the present invention to provide a syringe having an auto retractable surgical needle. It is a further object of the invention to provide a syringe which employs such an auto retractable needle in a sterile manner prior to use, and for permanently storing that surgical needle, once used, in a substantially safe manner. The invention may be utilised with so-called exchange needle syringes or with other syringes requiring auto retraction of the needle after use.

This invention is primarily intended to eliminate needle stick injuries, by causing the needle to retract automatically inside the barrel when the injection is complete thereby making the syringe safe to handle.

At the very least the invention provides an alternate means of accommodating the needle of a used syringe, including an exchange needle syringe, to protect against accidental injury arising from unwanted contact with the exposed needle once the syringe has been used.

Although the following description also generally refers to a syringe of conventional size, no such limitation is intended thereby, and reference to a syringe is meant to encompass any other needle/syringe combination including slimline syringes, where, by suitable adaptation, the invention may also be usefully applied.

### DISCLOSURE OF THE INVENTION

The invention in one broad form provides a safety syringe incorporating an auto retractable needle assembly therein, the safety syringe having a syringe barrel which has a hollow plunger slideably located therein, the hollow plunger having a proximal and distal end with piston means associated therewith at its distal end so as to be sealingly slideable within the barrel, the needle assembly comprising a needle and a needle hub, with a recess in the end of the barrel to hold the needle hub of the needle and a spring disposed over said needle hub and acting between the needle hub and the end of the barrel, the spring remaining under compression during use, the needle hub having sealing means associated therewith to prevent leakage between the needle hub and the end of the barrel, said needle hub having a groove or recess around its circumference in which a locking means mates so as to retain the needle hub in the end of the syringe barrel during use against the compressive action of the spring, and wherein said distal end of the hollow plunger has a sealing stopper, the plunger contacting in a first stage said needle assembly upon completion of an injection and when further depressed activating auto retraction of the needle hub by acting upon the locking means so as to cause said locking means to be dislodged from said groove or recess to release the needle hub by reaching a second stage, said sealing stopper dislodging from said distal end of the plunger when it reaches the second stage thereby releasing said needle hub and hence allowing the needle and spring to retract inside said hollow plunger under the influence of the spring.

Preferably the needle and needle hub are housed in a needle gland, which may be interchangeably connected to the barrel of the syringe.

Thus in a particularly preferred form of the invention, the invention in one broad form provides a safety syringe incorporating an auto retractable needle assembly therein, the safety syringe having a syringe barrel which has a hollow plunger slideably located therein, the hollow plunger having a proximal and distal end with piston means associated therewith at its distal end so as to be sealingly slideable within the barrel, the needle assembly comprising a gland bush, needle and needle hub, with a recess in the gland bush to hold a needle hub of the needle and a spring disposed over said needle hub and acting between the needle hub and the gland bush, the spring remaining under compression during use, the needle hub having sealing means associated therewith to prevent leakage between the needle hub and the gland bush, said needle hub having a groove around its circumference in which a locking means mates so as to retain the needle hub in the gland bush during use against the compressive action of the spring, and wherein said distal end of the hollow plunger has a sealing stopper, the plunger contacting in a first stage said needle assembly upon completion of an injection and when further depressed activating auto retraction of the needle hub by acting upon the needle assembly so as to cause said locking means associated with the needle gland to be dislodged from said groove to release the needle hub by reaching a second stage, said sealing stopper dislodging from said distal end of the plunger when it reaches the second stage thereby releasing said needle hub and hence allowing the needle and spring to retract inside said hollow plunger under the influence of the spring.

It will be understood that the needle gland allows the needle assembly to be interchangeably removed. However, the invention, remains the same, namely the use of a locking means located in either the barrel itself or in the gland when utilised, such that the action of the plunger causes the locking means to be released, thereby releasing the needle hub held in the barrel or gland as the case may be, so that the needle hub is caused to enter the hollow plunger when thus released.

The following description refers primarily to the use of a gland, however no limitation is intended thereby and any syringe is contemplated to the extent that where no gland is utilised, reference to gland should be taken to be a reference to the barrel itself, in which the gland would otherwise be held if one were utilised. In other words, the use of the gland in the barrel to provide interchangeability of the needle should be construed simply as a two part barrel structure, and where no gland is utilised, one might consider it as a simple integral structure.

Preferably the locking means is in the form of fingers or a full ring located about the internal periphery of the gland bush which mate (or mates as the case may be) with the groove located about the needle hub.

Alternatively the locking means may be independent of the gland bush and may include for example a dowel pin as described in one of the following embodiments.

The shape of the components such as the groove in the needle hub and the locking means, whether in the form of fingers or rings in the gland bush itself or an independent item such as dowel pin, may be of any suitable shape for the required purpose and include, but are not necessarily limited to such shapes as round, square and triangular.

Thus, preferably the needle assembly has means for removable attachment thereof to the end of the barrel, so as to provide a so-called exchange needle assembly for the syringe.

In this particular embodiment, preferably the means by which the needle assembly is removably attached to the barrel is provided by having the gland bush fitted to the end of the barrel by threaded means. Attachment means can be a standard screw fitting or a so-called luer-lock fitting.

Preferably, the piston means is slideable on the plunger to allow the plunger to move from the first stage to the second in order that auto retraction is activated. This movement is preferably achieved by having three ridges located about periphery of the plunger, two ridges only being located inside the piston seal at any one time. When extra pressure is exerted between the plunger and the barrel at completion of injection, the piston seal is forced up the plunger. This allows the plunger to protrude through the piston seal and pushes against the pre-assembled needle gland assembly to effect auto-retraction.

Alternatively the piston means may be simply compressible under increased pressure exerted at the end of the injection.

Preferably the needle hub is provided with a flange about its perimeter, and the inner wall of the recess in the gland bush is provided with a step, so that the spring is located between the flange and the step such that it is maintained in compression during use.

In one embodiment of the invention utilising a dowel pin, the plunger acts upon the needle hub directly to cause auto retraction, the dowel pin having a tapered end, and the groove around said needle hub being a tapered groove in which the said tapered end of dowel pin sits, so that on reaching the second stage, the plunger causes said needle hub to be pushed further into said recess, thereby pushing the tapered end of dowel pin out of the tapered groove, said sealing stopper dislodging from said distal end after completion of injection thereby allowing said needle hub and spring to retract inside said hollow plunger.

In this embodiment, the sealing means to prevent leakage between the needle hub and the gland bush is preferably a simple O-ring or similar seal located about the flange of the needle hub.

Preferably, the dowel pin is provided with means by which it is held in position once it is dislodged from the tapered groove, so as not to interfere with the retraction of the needle hub under the influence of the spring once the needle hub is released. Such means include an interference fit, eg by having a slight taper along the length of the dowel pin so that it is wedged in the housing after it is dislodged from the needle hub.

In an alternate embodiment, a sleeve is located about the needle hub and spring, the plunger acting upon the sleeve to cause auto retraction, the dowel pin having means by which it may be dislodged from the groove about the needle hub when the plunger reaches the second stage, the plunger causing said sleeve to be pushed further into said recess thereby dislodging said end of dowel pin out of said groove, said sealing stopper dislodging from said distal end after completion of injection allowing said needle hub and spring to retract inside said hollow plunger.

It will be understood in this embodiment that the pin need not have a tapered end, so that it will provide a more positive locking of the needle hub retaining it from movement in either direction whilst it is thus retained in the gland bush, ie prior to auto retraction.

Preferably in this embodiment, the dowel pin is provided with a tapered flange located on its side, against which the end of the sleeve acts so as to cause it to be dislodged to effect auto retraction. The advantage in this arrangement (ie utilising a sleeve located about the hub) is that the dowel pin is held back by positive means, ie under the influence of the sleeve, which means that the needle hub is free to retract without any possible interference from the dowel pin.

Preferably in this embodiment, the sealing means to prevent leakage between the needle hub and the gland bush is provided by a sealing washer which extends between the needle hub and the gland bush and hence across the sleeve surrounding the needle hub and spring, so that the action of the plunger in causing auto-retraction is as a result of the plunger acting indirectly on the sleeve through the sealing washer.

In an alternative embodiment to one described above utilising a dowel pin, the invention utilises fingers or a ring or other flange element located about the inner periphery of the gland bush.

In this case the needle assembly is preferably made up of the following parts:

### 1. Gland Bush

### 2. Sliding Spreader Bush

### 3. Gland Seal

### 4. Needle Hub

### 5. Compression Spring

In this embodiment, the sleeve described above in relation to the embodiment utilising a dowel pin, is instead in the form of a sliding spreader bush which is again located about the needle hub and spring, the plunger again acting in this case upon the sliding spreader bush to cause auto retraction, the fingers or ring located in the gland bush being able to be pushed apart and hence dislodged from the groove about the needle hub when the plunger reaches the second stage, the plunger causing said sliding spreader bush to be pushed further into said recess thereby dislodging said fingers or ring out of said groove, said sealing stopper once again dislodging from said distal end after completion of injection allowing said needle hub and spring to retract inside said hollow plunger.

It will be appreciated that in the case of a ring, there will need to be provided a region of discontinuity such as one or more slits or the like to allow the ring to spread.

Preferably the sliding spreader bush has a tapered region about its end to facilitate spreading the respective fingers or ring as the case may be, allowing for a smooth interaction. Conversely the portion of the gland bush which is engaged buy the sliding spreader bush to effect spreading of the fingers or ring is correspondingly tapered to facilitate a smooth interaction.

Once again, the advantage in this arrangement (ie utilising a sleeve in the form of sliding spreader bush located about the hub) is that the fingers or ring are held back by positive means, ie under the influence of the spreader bush, which means that the needle hub is free to retract without any possible interference from the fingers or ring.

Preferably in this embodiment, the sealing means to prevent leakage between the needle hub and the gland bush is again provided by a sealing washer which extends between the needle hub and the gland bush and hence across the spreader bush surrounding the needle hub and spring, so that the action of the plunger in causing auto-retraction is as a result of the plunger acting indirectly on the spreader bush sleeve through the sealing washer.

Thus, when the plunger is pushed down after injection the plunger contacts the seal. Pushing it down, causes the sliding spreader bush to be engaged with the fingers or ring formed on the inside of the gland bush. By continuing to push the plunger, the fingers or ring will be forced out which releases the needle hub. At the same time the sealing stopper, situated within the end of the hollow plunger, is dislodged inside the plunger and the compression spring forces the needle hub to retract inside the hollow plunger. Preferably two fingers are utilised in the gland bush.

Although the dowel arrangement is useful, it has been found that the use of two fingers in particular as described in this latter embodiment, makes assembly much easier and eliminates the more difficult dowel pin and tiny spring. Furthermore, the design may be more streamline, as here is no need to provide for the dowel pin with for example a clearance in the side of any cap utilised therewith.

In any of the foregoing embodiments, preferably a needle cap is provided with the assembly to protect the needle prior to use.

Prior to use, the needle cap protects the needle from damage and also acts as a safety cap for the medical staff using the syringe preventing needle stick injuries. The presence of the needle cap also means that the needle assembly may be conveniently changed in the case of an exchange needle embodiment without risk of needle stick injury which might occur if the needle were unprotected.

In the exchange needle case, the needle gland is preferably held in the needle cap by interference fit. Preferably there are matching splines on the outside of the needle gland that match with splines on the inside of needle cap.

The needle thus comes pre-assembled within a screw in gland bush and fitted with a safety cap to prevent needle stick injuries, so that all that is required is to remove the sterile packaging and screw the pre-assembled needle housing into the syringe and remove the safety cap. The syringe is then ready for use.

In this case, following assembly, ie after the needle gland is assembled with the needle inside and the protective needle cap is fitted, the assembly is screwed inside to the end of the barrel. A sealing point is secured by means of matching tapers between the needle gland and the inside of the threaded barrel.

After the needle gland assembly, ie with the needle hub fitted therein, is screwed into the barrel and the seal is reached, the needle cap is removed from the assembly making the needle ready for use. The added advantage of this syringe is that various size needles can be fitted allowing the health care profession to make a choice of which needle to use.

In any of the foregoing embodiments, the barrel of the syringe is preferably of clear plastic and is graduated to read fluid level.

The piston is preferably made of synthetic rubber and is fitted to the plunger so as to seal by way of an interference fit between the plunger and the barrel, thereby allowing the fluid to be extruded from the syringe, via the needle, in the usual manner.

### BRIEF DESCRIPTION OF THE DRAWINGS

The invention may be better understood from the following non-limiting description of a preferred embodiment, in which:

FIG. 1 is a cross sectional view of the needle gland assembly of a syringe according to a first embodiment of the invention,

FIG. 2 is a cross sectional view of the plunger for use with syringe according to a first embodiment of the invention,

FIG. 3 is a syringe barrel plunger for use with syringe according to a first embodiment of the invention,

FIG. 4 is a cross sectional view of an assembled syringe according to a second embodiment of the invention in which a sleeve is utilised, prior to use,

FIG. 5 is a cross sectional view of the syringe of FIG. 4, showing the plunger withdrawn after receiving fluid for an injection,

FIG. 6 is a cross sectional view of the syringe of FIGS. 4 and 5, showing the plunger depressed to the second stage after an injection to cause auto retraction of the needle,

FIG. 7 is a cross sectional view of the syringe of FIGS. 4 to 6 showing the retracted needle and spring in the hollow plunger,

FIG. 8 is a cross sectional view of the barrel of syringe (shown in its entirety in FIGS. 17 to 20), utilising internal fingers located in the gland bush and a sliding spreader sleeve syringe according to a further embodiment of the invention,

FIG. 9 is a cross sectional view of the plunger for use with the syringe of FIGS. 17 to 20,

FIG. 10 is the plan view of the proximal end of the plunger for use with the syringe of FIGS. 17 to 20,

FIG. 11 is a cross sectional side view of the end of the plunger of FIG. 10,

FIG. 12 is a cross sectional view of the cap for the syringe of FIGS. 17 to 20.

FIG. 13 is a cross sectional view of the needle gland for use with the syringe of FIGS. 18 to 21, the gland being able to be screwed into the end of the syringe barrel,

FIG. 14 is a cross sectional view of the needle hub for use with the syringe of FIGS. 18 to 21, and which is located in the needle gland of FIG. 13,

FIG. 15 is a cross sectional view of the sliding sleeve for use with the syringe of FIGS. 18 to 21, which is located about the needle hub of FIG. 14 and within the gland of FIG. 13,

FIG. 16 is a cross sectional view of the piston support which is mounted on the distal end of the plunger of FIG. 10 for use with the syringe of FIGS. 18 to 21,

FIG. 17 is a detailed cross section of the syringe depicted in FIGS. 18 to 21 in the region where the components of FIGS. 12 through 16 are utilised just prior to auto-retraction being effected,

FIG. 18 is cross sectional view of the entire syringe according to a further embodiment of the invention of which FIGS. 12 to 17 illustrate in detail various components and their interactions, the syringe having the plunger drawn back and filled with fluid ready for an injection,

FIG. 19 is a cross sectional view of the syringe of FIG. 18 showing the plunger depressed utilised just prior to auto-retraction being effected,

FIG. 20 is a cross sectional view of the syringe of FIG. 18 showing the plunger fully depressed to activate auto-retraction, and

FIG. 21 is a cross sectional view of the syringe of FIG. 18 showing auto-retraction having taken place, the needle hub now located in the hollow plunger.

### DETAILED DESCRIPTION OF THE DRAWINGS

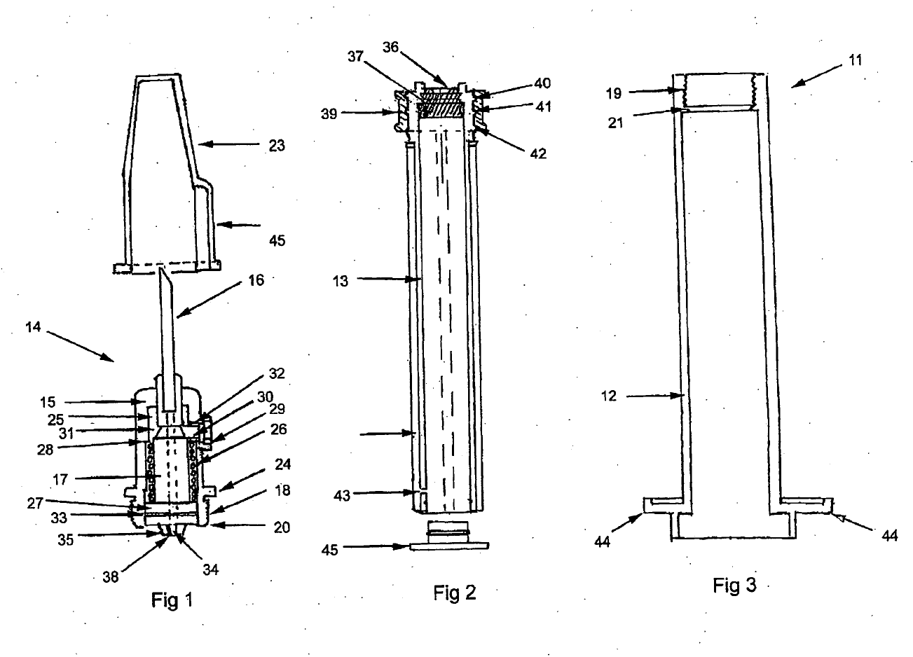

Referring generally to the FIGS. 1 to 3 there is shown the components of a syringe and needle combination generally referenced 11, which comprises a syringe barrel 12, a plunger 13 and a needle assembly generally referenced 14. The needle assembly 14 comprises a gland bush 15, which accommodates a needle 16 having a needle hub 17 at its upper end. The needle 16 protrudes through the gland bush 15. The needle hub 17 is preferably manufactured from medical grade plastic and supports the needle 16.

The gland bush 15 has a thread 18 about its periphery for mating engagement with a corresponding thread 19 located inside the end of the barrel 12. A male tapered portion 20 at the end of the gland bush 15 provides a seal against a corresponding female tapered portion 21 in the end of the barrel 12. The gland bush 15 thus creates a seal with the barrel 12 preventing leakage between the barrel 12 and the threaded portion 18 of the gland bush 15. The gland bush 15 is preferably made from plastic.

A safety cap 23 is preferably manufactured from medical grade plastic and fits neatly onto the gland bush 15. The safety cap 23 has on its outer surface ridges (not shown) to give finger grip to aid in its removable prior to use and to give drive when screwing the gland bush 15 into the body 12 of the syringe 11. The object of the safety cap 23 is to prevent needle stick injuries when fitting the needle assembly 14, ie by means of gland bush 15 to the syringe 11 and also to present the needle 16 sharp and sterile ready for use.

The gland bush 15 has a flange 24 adjacent the thread 18 to separate the threaded portion 18 from the region where the safety cap 23 sits.

The inside of the gland bush 15 has a recess 25 to hold the needle hub 17 about which is located a stainless steel spring 26. A flange 27 is located about the periphery of the needle hub 17, whilst a step 28 is provided in the inner wall of the recess 25 in gland bush 15. The spring 26 is maintained in compression between the flange 27 of needle hub 17 and the step 28 in the recess 25 of gland bush 15.

Also on the side of the gland bush 15 is a housing 29 to accommodate a dowel pin 30. A corresponding clearance region 45 is provided in the cap 23 to accommodate the housing 29.

Around the body of the needle hub 17 is a tapered groove 31. The end of the tapered dowel pin 30 sits in this groove 31. A small stainless steel spring 32 urges the dowel pin 30 into locking engagement with groove 31 in the needle hub 17.

As the plunger 13 comes into play at the completion of injection, the needle hub 17 is pushed further into the recess 25 of the needle gland bush 15 causing the dowel pin 30 to be pushed outwardly, causing auto retraction of the needle 16, by virtue of the spring 26 operating on the released hub 17, since this allows the spring 26 to force the needle 16 into the retracted position. A slight taper is provided along the length of the dowel pin 30 to keep it tightly in the housing 29 when it is withdrawn from the groove 31.

The needle hub 17 has around the flange 27, a seal 33 to prevent leakage between the hub 17 and the recess 25 inside the gland 15.

A hole 34 is provided through the middle of needle hub 17 to allow the passage of fluid being injected through needle 16 and into the patient.

A spigot 35 is located on the bottom of the needle hub 17 to align the end of the needle hub 17 with a female spigot 36 located in the inner seal or sealing stopper 37 in the end of plunger 13. The sealing stopper 37 seals the inside of the plunger 13 prior to final injection being completed.

A groove 38 allows the last of the injected fluid between the needle hub 17 and the plunger 13 to escape through the needle 16.

The plunger 13 is preferably manufactured from clear medical grade plastic and has clipped on the outside a piston seal 39 The piston seal 39 is allowed to move on final injection in order to activate the auto retract facility by pushing down on the needle hub 17 as described above. This movement is achieved by having three ridges 40, 41, and 42 on the plunger 13. Two ridges only are located inside the piston seal 39 at any one time. When extra pressure is exerted between the plunger 13 and the barrel 12 at completion of injection, the piston seal 39 is forced up the plunger 13, ie the piston seal 39 moves from ridges 40, 41 to ridges 41, 42. This allows the plunger 13 to protrude through the piston seal 39 and pushes against the pre-assembled needle gland assembly 14, in this case pushing directly on needle hub 17.

The plunger 13 is hollow, having a thumb cap 45 located at its proximal end. The plunger 13 is hollow to take the needle hub 17 and hence needle 16 as well as stainless steel spring 26 after injection is complete. The sealing stopper 37 is thus dislodged when injection is complete by contact with the spigot 35 on the needle hub 17. The sealing stopper 37 is forced up inside the plunger 13 by the retracting needle hub 17 and the stainless steel compression spring 16. The retraction of the sealing stopper 37 is slowed in its travel by the sealing surface inside the barrel 12 blocking air that is only allowed to escape slowly through an exhaust hole 43 at the top of the plunger. This slow retraction prevents splash from the needle end 16.

A slow retraction can also be achieved by using a slightly tapered hollow region inside the plunger 13. As the sealing stopper returns 37 inside the plunger 13, after injection, the air can escape in front of the stopper seal 37 in increasing amounts. This will allow the needle 16 and stopper 37 to return slowly at first then increase in velocity as the air escapes while the spring 26 produces the movement of the needle 26 and sealing stopper 37 upwards inside the plunger 13.

The barrel 12 thus holds the plunger 13 containing the piston seal 39 and the sealing stopper 37. The barrel 13 is made from clear medical grade plastic and is calibrated to give visual levels of quantity of fluid to be injected. The gland bush 15 containing the needle cap 23 and needle 16, being a pre-assembled unit, screws into the barrel 12. Two protrusions 44 at the top of the barrel are finger grips to allow injection in the usual manner.

In use, the syringe 11 is removed from sterile packaging. The pre-assembled gland assembly 14 is removed from sterile packaging and by holding by the safety cap 23, the gland assembly 14 is screwed into the threaded end 19 of the syringe barrel 12. After removing the cap 23, the needle 16 is inserted into the vial. The syringe 11 is filled by drawing back the plunger 13. The injection is given. At the completion of injection, the plunger 13 is forced down until the needle hub 17 moves down and releases the dowel pin 30 and the sealing stopper 37. At this point, the needle 16 retracts inside the plunger 13, and hence inside the barrel 12. The syringe 11 is now safe and inoperable. The whole syringe 11 may now be safely disposed of in a responsible manner. A needle change can be made after filling the syringe 11 from a vial prior to injection if required.

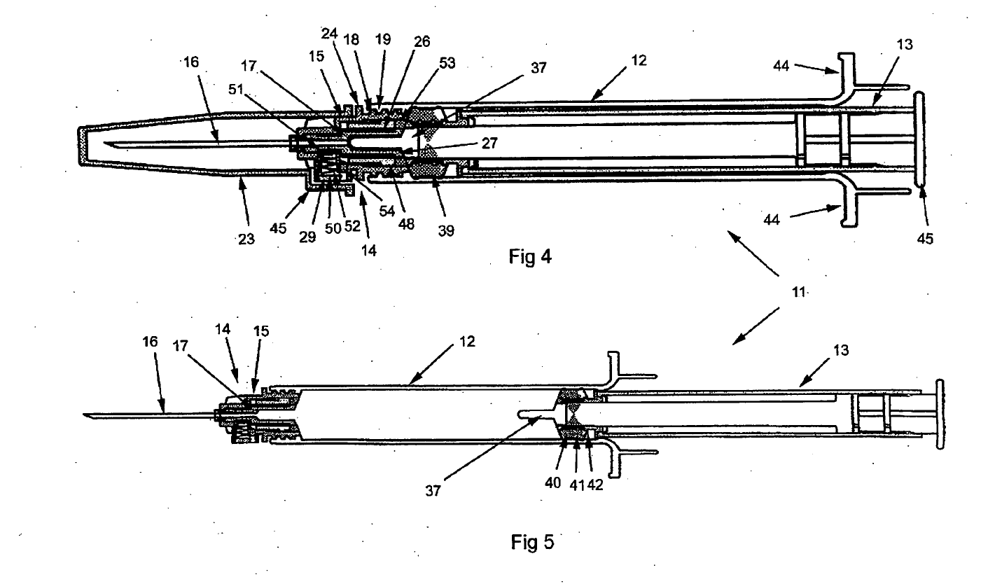

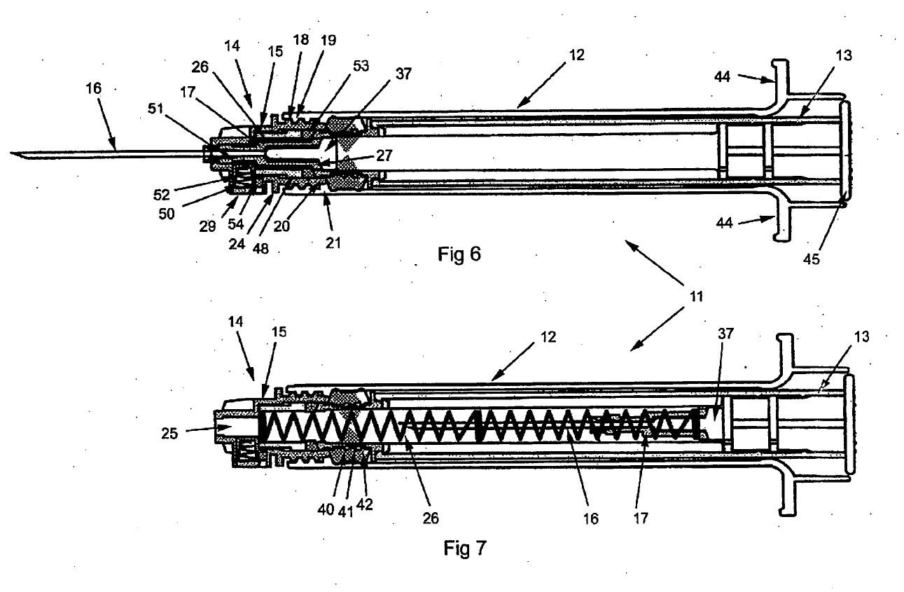

Referring generally to FIGS. 4 to 7, there is illustrated a syringe generally referenced 11 wherein each of the components, is referenced with the same reference numerals as those in FIG. 1 to 3, where the components are substantially identical.

In this embodiment however the end of the plunger 13 does not act directly on the needle hub 17 as is the case with the embodiment shown in FIGS. 1 to 3, but rather there is a sleeve 48 located about both the spring 26 and the needle hub 17. A seal 49 extends across the gap between the needle hub 17 and the inner wall of the gland bush 15. In this case the plunger 13 effects auto retraction by acting upon the sleeve 48, via the seal 49. Dowel pin 50 is urged into mating contact with the groove 51 by means of spring 52. The pin 50 is provided with a tapered flange 54 on its side against which the end of the sleeve 48 presses when the plunger 13 is depressed at the completion of the injection, ie as it moves from the first stage to the second stage as discussed above.

Thus in FIGS. 4 to 7 there is shown a syringe and needle combination generally referenced 11, which comprises a syringe barrel 12, a plunger 13 and a needle assembly generally referenced 14. The needle assembly 14 comprises a gland bush 15, which accommodates a needle 16 having a needle hub 17 at its upper end. The needle 16 protrudes through the gland bush 15. The needle hub 17 is preferably manufactured from medical grade plastic and supports the needle 16.

The gland bush 15 has a thread 18 about its periphery for mating engagement with a corresponding thread 19 located inside the end of the barrel 12. A male tapered portion 20 at the end of the gland bush 15 provides a seal against a corresponding female tapered portion 21 in the end of the barrel 12. The gland bush 15 thus creates a seal with the barrel 12 preventing leakage between the barrel 12 and the threaded portion 18 of the gland bush 15. The gland bush 15 is preferably made from plastic.

A safety cap 23 is preferably manufactured from medical grade plastic and fits neatly onto the gland bush 1S. The safety cap 23 has on its outer surface ridges (not shown) to give finger grip to aid in its removable prior to use and to give drive when screwing the gland bush 15 into the body 12 of the syringe 11. The object of the safety cap 23 is to prevent needle stick injuries when fitting the needle assembly 14, ie by means of gland bush 15 to the syringe 11 and also to present the needle 16 sharp and sterile ready for use.

The gland bush 15 has a flange 24 adjacent the thread 18 to separate the threaded portion 18 from the region where the safety cap 23 sits.

The inside of the gland bush 15 has a recess 25 to hold the needle hub 17 about which is located a stainless steel spring 26. A flange 27 is located about the periphery of the needle hub 17, whilst a step 28 is provided in the inner wall of the recess 25 in gland bush 15. The spring 26 is maintained in compression between the flange 27 of needle hub 17 and the step 28 in the recess 25 of gland bush 15.

Also on the side of the gland bush 15 is a housing 29 to accommodate the dowel pin 50. A corresponding clearance region 45 is provided in the cap 23 to accommodate the housing 29.

Around the body of the needle hub 17 is a groove 51. The end of the dowel pin 50 sits in groove 51. A small stainless steel spring 52 urges the dowel pin 50 into locking engagement with groove 51 in the needle hub 17.

Between the needle hub 17 and the wall of the gland bush 15 the a seal 53 to prevent leakage between the hub 17 and the recess 25 inside the gland 15.

As the plunger 13 comes into play at the completion of injection, the plunger acts on the seal 53 so that sleeve 48 is pushed further into the recess 25 of the needle gland bush 15 causing the dowel pin 50 to be pushed outwardly by virtue of the action of the end of the sleeve on the tapered surface of the flange 54 on the side of the pin 50, causing auto retraction of the needle 16, by virtue of the spring 26 operating on the released hub 17, since this allows the spring 26 to force the needle 16 into the retracted position.

A sealing stopper 37 seals the inside of the plunger 13 prior to final injection being completed.

The plunger 13 is preferably manufactured from clear medical grade plastic and has clipped on the outside a piston seal 39 The piston seal 39 is allowed to move on final injection in order to activate the auto retract facility by pushing down on the sleeve 48, via seal 53 as described above. This movement is achieved by having three ridges 40, 41, and 42 on the plunger 13. Two ridges only are located inside the piston seal 39 at any one time. When extra pressure is exerted between the plunger 13 and the barrel 12 at completion of injection, the piston seal 39 is forced up the plunger 13, ie the piston seal 39 moves from ridges 40, 41 to ridges 41, 42. This allows the plunger 13 to protrude through the piston seal 39 and pushes against the pre-assembled needle gland assembly 14, in this case pushing indirectly on sleeve 48 via seal 53.

The plunger 13 is hollow, having a thumb cap 45 located at its proximal end. The plunger 13 is hollow to take the needle hub 17 and hence needle 16 as well as stainless steel spring 26 after injection is complete. The sealing stopper 37 is thus dislodged when injection is complete. The sealing stopper 37 is forced up inside the plunger 13 by the retracting needle hub 17 and the stainless steel compression spring 16. The retraction of the sealing stopper 37 may be slowed as described above in relation to the embodiment of FIG. 1, to prevent splash from the needle end 16.

The barrel 12 thus holds the plunger 13 containing the piston seal 39 and the sealing stopper 37. The barrel 13 is made from clear medical grade plastic and is calibrated to give visual levels of quantity of fluid to be injected. The gland bush 15 containing the needle cap 23 and needle 16, being a pre-assembled unit, screws into the barrel 12. Two protrusions 44 at the top of the barrel are finger grips to allow injection in the usual manner.

In use, the syringe 11 is removed from sterile packaging. The pre-assembled gland assembly 14 is removed from sterile packaging and by holding by the safety cap 23, the gland assembly 14 is screwed into the threaded end 19 of the syringe barrel 12 as shown in FIG. 2. After removing the cap 23, the needle 16 is inserted into the vial. The syringe 11 is filled by drawing back the plunger 13 as shown in FIG. 3. The injection is given. At the completion of injection, as shown in FIG. 4, the plunger 13 is forced down until the sleeve 48 moves down and releases the dowel pin 50 and the sealing stopper 37. At this point, the needle 16 retracts inside the plunger 13, and hence inside the barrel 12 as shown in FIG. 5. The syringe 11 is now safe and inoperable. The whole syringe 11 may now be safely disposed of in a responsible manner. A needle change can be made after filling the syringe 11 from a vial prior to injection if required.

The advantages of the invention in either embodiment, lie in the locking dowel and the pre-assembled gland. The invention also provides slow retraction of the needle hub by controlling the exhaust of air. With advantage, the location of the plunger sealing stopper to needle hub is facilitated by the use of the spigot. Using a movable piston allows complete exhaustion of fluid at completion of injection while allowing the plunger to travel further to execute needle retraction.

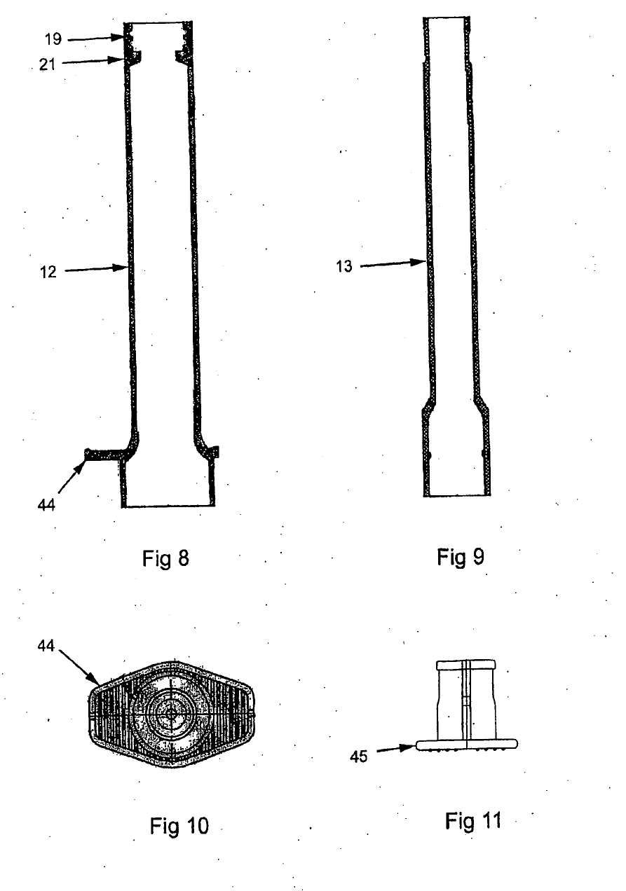

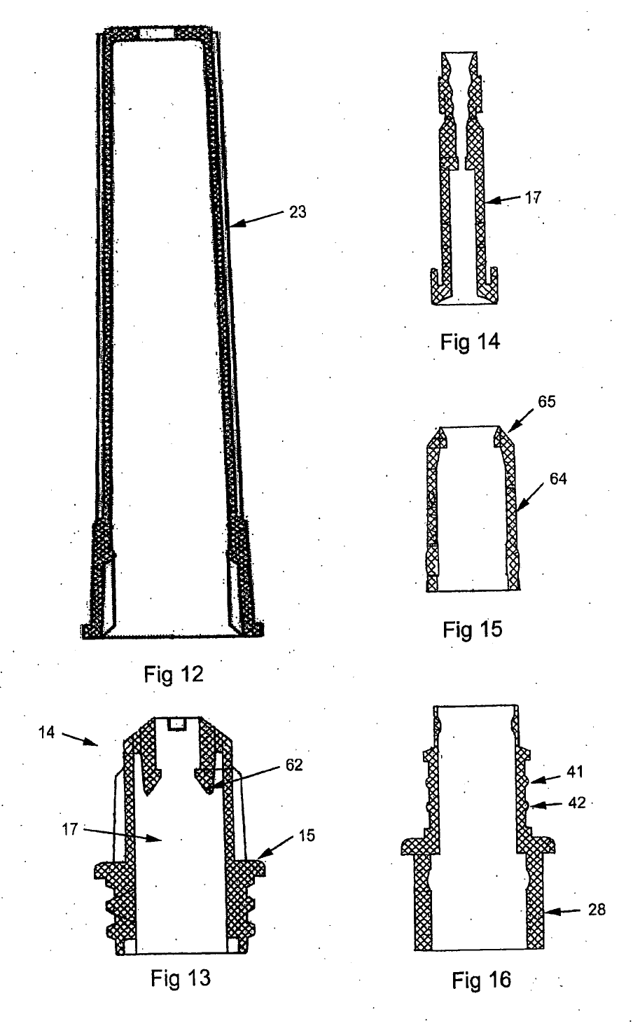

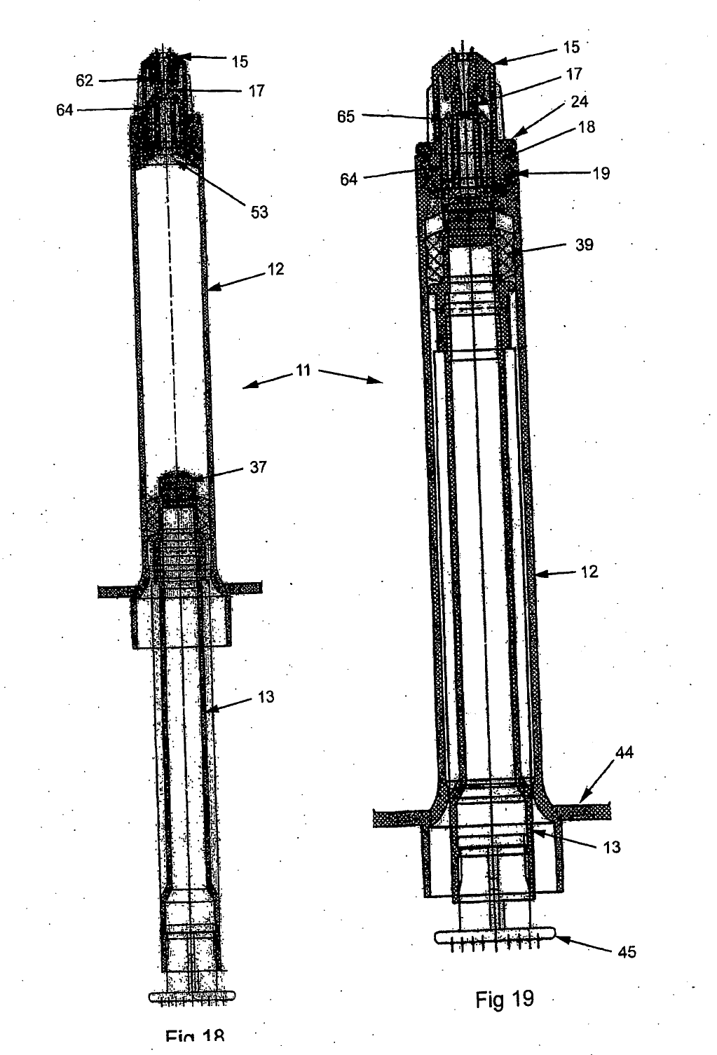

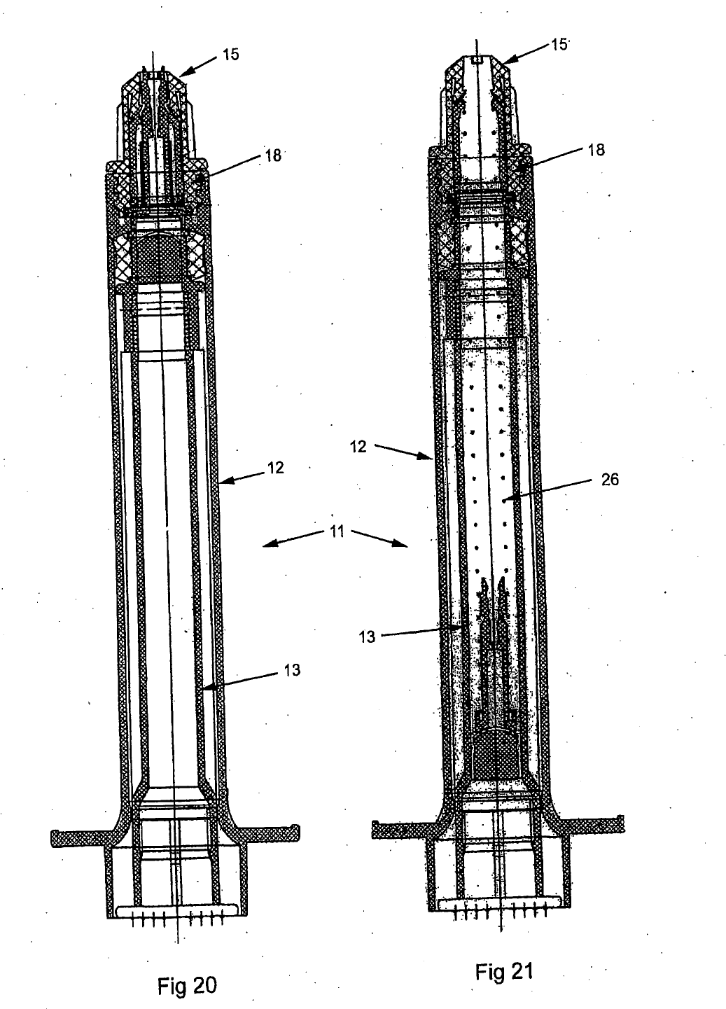

Turning to FIGS. 8 to 21, there is shown a further embodiment of the invention in which like components to those already described utilise the same reference numerals.

The sliding spreader bush 64 has tapered leading end 65.

The gland bush 15 is shown so that the shape of the fingers 62 is discernible.

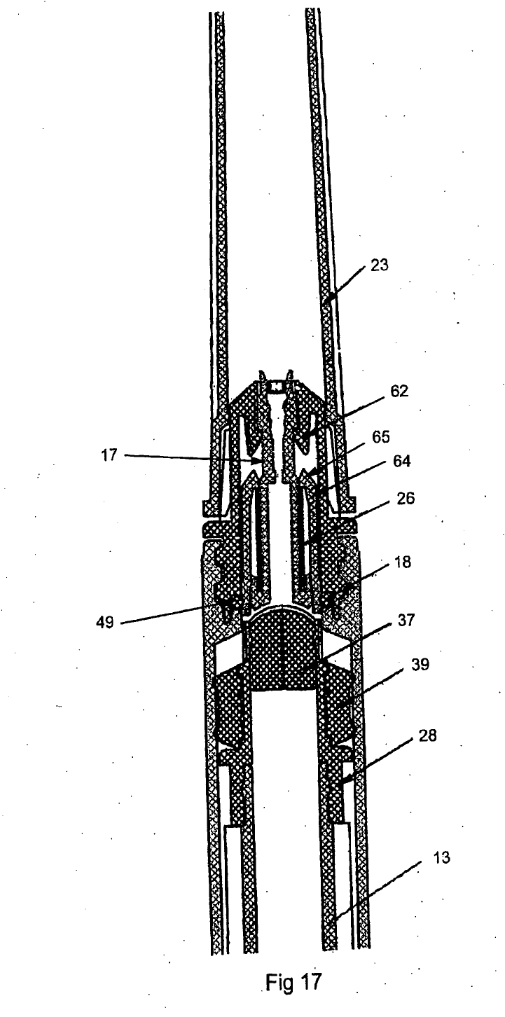

Referring to FIGS. 17 to 21, when the plunger 13 (not shown) is pushed down after injection the plunger 13 contracts the seal 53 pushing it down. This will cause the sliding spreader bush 64 to be engaged with the two fingers 62 cast on the inside of the gland bush 15. By continuing to push the plunger 13 the two fingers 62 will be forced out which releases the needle hub 17. At the same time the sealing stopper 36 (not shown), situated within the end of the hollow plunger 13, is dislodged inside the plunger 13 and the compression spring 26 forces the needle hub 17 to retract inside the hollow plunger 13.

The other aspects of this embodiment's overall operation are akin to those described above in relation to FIGS. 1 to 3 and FIGS. 4 to 7.

It will be appreciated by those skilled in the art that many modifications and variations may be made to the embodiments described herein without departing from the spirit or scope of the invention.

Throughout the specification the word “comprise” and its derivatives are intended to have an inclusive rather than exclusive meaning unless the context requires otherwise.

1. An auto-retractable safety syringe incorporating an auto retractable needle assembly therein, the safety syringe having a syringe barrel which has a hollow plunger slideably located therein, the hollow plunger having a proximal and distal end with piston means associated therewith at its distal end so as to be sealingly slideable within the barrel, the needle assembly comprising a needle and a needle hub, with a recess in the end of the barrel to hold the needle hub of the needle and a spring disposed over said needle hub and acting between the needle hub and the end of the barrel, the spring remaining under compression during use, the needle hub having sealing means associated therewith to prevent leakage between the needle hub and the end of the barrel, said needle hub having a groove or recess around its circumference in which a locking means mates so as to retain the needle hub in the end of the syringe barrel during use against the compressive action of the spring, and wherein said distal end of the hollow plunger has a sealing stopper, the plunger contacting in a first stage said needle assembly upon completion of an injection and when further depressed activating auto retraction of the needle hub by acting upon the locking means so as to cause said locking means to be dislodged from said groove or recess to release the needle hub by reaching a second stage, said sealing stopper being dislodged from said distal end of the plunger when it reaches the second stage by the action of the needle as it retract, thereby releasing said needle hub and hence allowing the needle and spring to retract inside said hollow plunger under the influence of the spring.

2. An auto-retractable safety syringe according to claim 1 in which the needle and needle hub are housed in a needle gland, which may be interchangeably connected to the barrel of the syringe.

3. An auto-retractable safety syringe according to claim 2, in which the means by which the needle assembly is removably attached to the barrel is provided by having the gland bush fitted to the end of the barrel by threaded means.

4. An auto-retractable safety syringe according to claim 3, in which attachment is by means of a standard screw fitting or a so-called luer-lock fitting.

5. An auto-retractable safety syringe according to claim 2, the safety syringe having a syringe barrel which has a hollow plunger slideably located therein, the hollow plunger having a proximal and distal end with piston means associated therewith at its distal end so as to be sealingly slideable within the barrel, the needle assembly comprising a gland bush, needle and needle hub, with a recess in the gland bush to hold a needle hub of the needle and a spring disposed over said needle hub and acting between the needle hub and the gland bush, the spring remaining under compression during use, the needle hub having sealing means associated therewith to prevent leakage between the needle hub and the gland bush, said needle hub having a groove around its circumference in which a locking means mates so as to retain the needle hub in the gland bush during use against the compressive action of the spring, and wherein said distal end of the hollow plunger has a sealing stopper, the plunger contacting in a first stage said needle assembly upon completion of an injection and when further depressed activating auto retraction of the needle hub by acting upon the needle assembly so as to cause said locking means associated with the needle gland to be dislodged from said groove to release the needle hub by reaching a second stage, said sealing stopper dislodging from said distal end of the plunger when it reaches the second stage thereby releasing said needle hub and hence allowing the needle and spring to retract inside said hollow plunger under the influence of the spring.

6. An auto-retractable safety syringe according claim 5 in which the gland is integral with the syringe barrel and is therefore not interchangeable but forms a part of the barrel.

7. An auto-retractable safety syringe according to claim 1 in which the locking means is in the form of fingers or a full ring located about the internal periphery of the gland bush which mate (or mates as the case may be) with the groove located about the needle hub.

8. An auto-retractable safety syringe according to claim 1 in which the locking means is independent of the gland bush or the barrel of the syringe as the case may be, and includes a dowel pin.

9. An auto-retractable safety syringe according to claim 1, in which the shape of the components including the groove in the needle hub and the locking means, whether in the form of fingers or rings in the gland bush itself or the syringe barrel as the case may be, or an independent item such as dowel pin, may be of any suitable shape for the required purpose and include, but are not necessarily limited to such shapes as round, square and triangular.

10. An auto-retractable safety syringe according to claim 1 in which the piston means is slideable on the plunger to allow the plunger to move from the first stage to the second in order that auto retraction is activated.

11. An auto-retractable safety syringe according claim 10 in which the movement of the piston is achieved by having three ridges located about periphery of the plunger, two ridges only being located inside the piston seal at any one time, so that when extra pressure is exerted between the plunger and the barrel at completion of injection, the piston seal is forced up the plunger, thereby allowing the plunger to protrude through the piston seal and pushes against the pre-assembled needle gland assembly to effect auto-retraction.

12. An auto-retractable safety syringe according to claim 10 in the piston means is simply compressible under increased pressure exerted at the end of the injection.

13. An auto-retractable safety syringe according to claim 1 in which the needle hub is provided with a flange about its perimeter, and the inner wall of the recess in the gland bush is provided with a step, so that the spring is located between the flange and the step such that it is maintained in compression during use.

14. An auto-retractable safety syringe according to claim 1 in which a dowel pin is utilised, the plunger acts upon the needle hub directly to cause auto retraction, the dowel pin having a tapered end, and the groove around said needle hub being a tapered groove in which the said tapered end of dowel pin sits, so that on reaching the second stage, the plunger causes said needle hub to be pushed further into said recess, thereby pushing the tapered end of dowel pin out of the tapered groove, said sealing stopper dislodging from said distal end after completion of injection thereby allowing said needle hub and spring to retract inside said hollow plunger.

15. An auto-retractable safety syringe according to claim 14 in which the sealing means to prevent leakage between the needle hub and the gland bush is preferably a simple O-ring or similar seal located about the flange of the needle hub.

16. An auto-retractable safety syringe according to claim 15 in which the dowel pin is provided with means by which it is held in position once it is dislodged from the tapered groove, so as not to interfere with the retraction of the needle hub under the influence of the spring once the needle hub is released.

17. An auto-retractable safety syringe according to claim 16 in which the means by which the dowel pin is held in position include an interference fit, such as having a slight taper along the length of the dowel pin so that it is wedged in the housing after it is dislodged from the needle hub.

18. An auto-retractable safety syringe according to claim 1, wherein a sleeve is located about the needle hub and spring, the plunger acting upon the sleeve to cause auto retraction, the dowel pin having means by which it may be dislodged from the groove about the needle hub when the plunger reaches the second stage, the plunger causing said sleeve to be pushed further into said recess thereby dislodging said end of dowel pin out of said groove, said sealing stopper dislodging from said distal end after completion of injection allowing said needle hub and spring to retract inside said hollow plunger.

19. An auto-retractable safety syringe according to claim 18 in which the pin does not have a tapered end, so that it will provide a more positive locking of the needle hub retaining it from movement in either direction whilst it is thus retained in the gland bush, ie prior to auto retraction.

20. An auto-retractable safety syringe according to claim 19 the dowel pin is provided with a tapered flange located on its side, against which the end of the sleeve acts so as to cause it to be dislodged to effect auto retraction.

21. An auto-retractable safety syringe according to claim 18, the sealing means to prevent leakage between the needle hub and the gland bush is provided by a sealing washer which extends between the needle hub and the gland bush and hence across the sleeve surrounding the needle hub and spring, so that the action of the plunger in causing auto-retraction is as a result of the plunger acting indirectly on the sleeve through the sealing washer.

22. An auto-retractable safety syringe according to claim 1 in which fingers or a ring or other flange element located about the inner periphery of the gland bush is utilised, the needle assembly comprising a gland bush, sliding spreader bush, gland seal, needle hub and compression spring, wherein the sleeve is in the form of a sliding spreader bush which is located about the needle hub and spring, the plunger acting upon the sliding spreader bush to cause auto retraction, the fingers or ring located in the gland bush being able to be pushed apart and hence dislodged from the groove about the needle hub when the plunger reaches the second stage, the plunger causing said sliding spreader bush to be pushed further into said recess thereby dislodging said fingers or ring out of said groove, said sealing stopper once again dislodging from said distal end after completion of injection allowing said needle hub and spring to retract inside said hollow plunger.

23. An auto-retractable safety syringe according claim 22 in which in the case of a ring, there will need to be provided a region of discontinuity such as one or more slits or the like to allow the ring to spread.

24. An auto-retractable safety syringe according to claim 22 in which the sliding spreader bush has a tapered region about its end to facilitate spreading the respective fingers or ring as the case may be, allowing for a smooth interaction.

25. An auto-retractable safety syringe according to claim 22 in which the portion of the gland bush which is engaged by the sliding spreader bush to effect spreading of the fingers or ring is correspondingly tapered to facilitate a smooth interaction.

26. An auto-retractable safety syringe according to claim 21 the sealing means to prevent leakage between the needle hub and the gland bush is again provided by a sealing washer which extends between the needle hub and the gland bush and hence across the spreader bush surrounding the needle hub and spring, so that the action of the plunger in causing auto-retraction is as a result of the plunger acting indirectly on the spreader bush sleeve through the sealing washer.

27. An auto-retractable safety syringe according to claim 26 wherein when the plunger is pushed down after injection the plunger contacts the seal, pushing it down, causes the sliding spreader bush to be engaged with the fingers or ring formed on the inside of the gland bush, and by continuing to push the plunger, the fingers or ring will be forced out which releases the needle hub, whilst at the same time the sealing stopper, situated within the end of the hollow plunger, is dislodged inside the plunger and the compression spring forces the needle hub to retract inside the hollow plunger.

28. An auto-retractable safety syringe according to claim 27 wherein two fingers are utilised in the gland bush.

29. An auto-retractable safety syringe according to claim 1 in which a needle cap is provided with the assembly to protect the needle prior to use.

30. An auto-retractable safety syringe according to claim 29 wherein the needle gland is preferably held in the needle cap by interference fit. Preferably there are matching splines on the outside of the needle gland that match with splines on the inside of needle cap.

31. An auto-retractable safety syringe according to claim 1 in which the barrel of the syringe is preferably of clear plastic and is graduated to read fluid level.

32. An auto-retractable safety syringe according to claim 1 in which the piston is preferably made of synthetic rubber and is fitted to the plunger so as to seal by way of an interference fit between the plunger and the barrel, thereby allowing the fluid to be extruded from the syringe, via the needle, in the usual manner.

33. (canceled)
